(19) United States
(12) Patent Application Publication
(10) Pub. No.: US 2023/0190736 A1
(43) Pub. Date: Jun. 22, 2023

(54) SMALL MOLECULE THERAPEUTICS FOR THE TREATMENT OF VIRAL INFECTIONS

(71) Applicant: The Regents of the University of California, Oakland, CA (US)

(72) Inventors: Vaithilingaraja Arumugaswami, Los Angeles, CA (US); Heather R. Christofk, Los Angeles, CA (US); Robert Damoiseaux, Los Angeles, CA (US); Gustavo Garcia, Los Angeles, CA (US); Peter Mullen, Los Angeles, CA (US); Brigitte N. Gomperts, Los Angeles, CA (US)

(21) Appl. No.: 17/917,445

(22) PCT Filed: Apr. 9, 2021
(86) PCT No.: PCT/US21/26627

§ 371 Date: Oct. 6, 2022

Related U.S. Application Data
(60) Provisional application No. 63/148,445, filed on Feb. 11, 2021.
(60) Provisional application No. 63/056,315, filed on Jul. 24, 2020.
(60) Provisional application No. 63/008,231, filed on Apr. 10, 2020.

(51) Int. Cl. A61K 31/497; A61K 45/06; A61P 31/14; A61K 31/5377; A61K 31/436; A61K 31/4706

(52) U.S. Cl. CPC A61K 31/497; A61K 45/06; A61P 31/14; A61K 31/5377; A61K 31/436; A61K 31/4706

(57) ABSTRACT

This disclosure relates to methods of treating viral infections, such as influenza or coronavirus, with certain inhibitors, such as kinase inhibitors.

### RELATED APPLICATIONS

This application claims the benefit of U.S. Provisional Patent Application No. 63/148,445, filed on Feb. 11, 2021; U.S. Provisional Patent Application No. 63/056,315, filed Jul. 24, 2020; and U.S. Provisional Patent Application No. 63/008,231, filed Apr. 10, 2020. The contents of each of the foregoing applications are hereby incorporated by reference in their entirety.

### BACKGROUND

Viral infections continue to represent a significant threat to humanity. For example, Coronavirus disease (COVID-19) is an infectious disease caused by a newly discovered coronavirus, severe acute respiratory syndrome-related coronavirus 2 (SARS-CoV-2). COVID-19 is currently causing pandemic and endangering lives globally. To date, the disease has spread to 219 countries or territories, and the number of COVID-19 cases has surpassed 132,847,000 globally, with over 2,879,787 deaths. Furthermore, to limit the spread of the virus, multiple countries have been forced to adopt social distancing policies, which have resulted in severe economic damage.

SARS-CoV-2 is an enveloped positive-sense single-stranded RNA virus that enters its host cell by binding to the angiotensin converting enzyme 2 (ACE2) receptor that is expressed on both alveolar type 1 and 2 cells in the lung. However, type 2 alveolar cells (AT2) have the most ACE2 expression of the cell types in the lung with one study showing 83% of AT2 cells with ACE expression. The major causes of morbidity and mortality from COVID-19 are acute lung injury with diffuse alveolar damage resulting in acute respiratory distress syndrome. This results from viral replication in lung epithelial cells causing cell injury, death and a vigorous immune response.

Despite the significant issues posed by viral infections, such as COVID-19, there are few methods for treating and preventing the infections. For instance, an effective cure for COVID-19 and other coronaviruses has yet to be developed. Accordingly, there is an ongoing need for new methods of treating viral infections.

### SUMMARY OF THE INVENTION

In certain aspects, the present disclosure provides methods of treating a viral infection in a subject in need thereof, comprising administering a compound selected from a PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, an mTORC inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

In certain aspects, the present disclosure provides compositions comprising an compound and a pharmaceutically acceptable excipient, wherein the composition is formulated for inhalation; and the compound is selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, an mTORC inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

In certain aspects, the present disclosure provides inhalers comprising a composition of the disclosure.

### BRIEF DESCRIPTION OF THE DRAWINGS

FIG. 1A depicts an infectious SARS-CoV-2 cell culture system, in particular bright field images of Vero-E6 cells infected with SARS-CoV-2 virus. Viral cytopathic effects are noted in the infected culture. 10× magnification.

FIG. 1B depicts an infectious SARS-CoV-2 cell culture system, in particular, ICC images of infected cells stained for spike antigen. Mouse monoclonal antibody (MS Ab) and a guinea pig antibody (GP) targeting SARS-CoV spike protein were used. Mock control cells were stained with MS Ab. 20× magnification.

FIG. 1C depicts an infectious SARS-CoV-2 cell culture system subjected to antiviral compound testing. ICC images show that hydroxychloroquine drug efficiently inhibited SARS-CoV-2 infection. The mock and infected cells were immunostained with dsRNA antibody, which recognizes double strand RNA generated during viral replication. 20× magnification.

FIG. 2 shows an exemplary workflow for the high throughput screen.

FIGS. 3A & B depicts bright field microscopic images of SARS-CoV-2 infected cells after treatment by exemplary compounds of the present disclosure, showing no or reduced level of viral CPE. DMSO Vehicle treated cells had pronounced viral CPE.

FIG. 4 shows a connectivity map of exemplary anti-SARS-CoV-2 compounds and their cellular targets.

FIG. 5 depicts an immunofluorescent assay to define the IC₅₀ of drug compounds exhibiting anti SARS-CoV-2 activity in secondary drug screen. Berzosertib (VE-822) and Vistusertib (AZD2014) are at IC₅₀ of 25 nM. Nilotinib, NVP-BHG712, VPS34-INI and YM201636 are at IC₅₀ ranges between 250 nM-500 nM.

FIG. 6 shows a connectivity map of exemplary anti-SARS-CoV-2 compounds and their cellular targets.

FIGS. 7A & B show that viruses dysregulate mTORC1. FIG. 7A shows that mTORC1 regulates cell growth by increasing the activity or expression of enzymes in anabolic pathways. FIG. 7B shows that the adenovirus activates mTORC1, shown by increased pS6K. MCF10A cells were seeded and allowed to adhere to the plate overnight. After 24 hours the cells were infected or mock infected with adenovirus for 22 hours. After 22 hours infection, the media was replaced with either vehicle media or media containing methionine. Cells were then harvested after 2 hours and immunoblotted. Activity is not reduced even when amino acids, such as methionine, are removed from the media.

FIG. 8 shows that SARS-CoV-2 activates mTORC1. Human lung stem cells were grown as air liquid interface cultures. These models mimic the cell types and architecture of the human lung and are readily infected by SARS-CoV-2. Donor 1 had healthy lungs, whereas Donor 2 had fibrosis in their lungs. Basal airway stem cells obtained from lung donors were used to generate primary human air-liquid interface cultures over a 28-day period. When fully differentiated, the cells were infected with SARS-CoV-2 for 3 days. The cells were then harvested and immunoblotted. It was confirmed that mTORC1 activity was increased by SARS-CoV-2 infection in cells from both donors by observing increased pS6 and/or p4E-BP1 levels in the infected cells.

We used airway basal stem cells obtained from lung donors to generate primary human air-liquid interface cultures over a 28-day period. When fully differentiated, we infected the ALI cultures with SARS-CoV-2 for 3 days. We then harvested protein and immunoblotted for the indicated proteins.

FIG. 9 shows that mTORC1 inhibition reduces SARS-CoV-2 replication. Vero cells were seeded and allowed to adhere to the plate overnight. After 24 hours, the cells were mock infected or infected with SARS-CoV-2 and treated with the indicated inhibitors. After 72 hours, the cells were fixed and stained with DAPI (dark grey) and an antibody to SARS-CoV-2 spike protein (light grey). Images were obtained using fluorescence microscopy.

FIG. 10 shows that berzosertib is safe for human lung ALI cells even at 50 concentration. Graph shows luminescence value (Y-axis) of berzosertib treated cell. Cells were treated with berzosertib at various concentrations and assay was performed 48 h post-treatment. Series 1 and 2 are the values from independent wells.

FIGS. 11A & B show berzosertib antiviral activity in lung epithelial cells. FIG. 11A depicts a graph showing the 8 dose-response curve of berzosertib in SARS-CoV-2 (MOI 0.1) infected human primary lung ALI culture at 48 hours. FIG. 11B shows immunofluorescent images showing dose-dependent reduction of SARS-CoV-2 replication in berzosertib treated ALI culture. SARS-CoV-2 positive cells were quantified to estimate the percent inhibition. Graph shows over 70% (dotted horizontal line) inhibition of SARS-CoV-2 following berzosertib treatment.

### DETAILED DESCRIPTION OF THE INVENTION

COVID-19, which is a coronavirus is the causative agent of a lethal infection that also induces many co-morbidities in survivors, such as lung scarring. Currently, there is no approved therapy for the treatment of coronavirus infection.

The strategy for developing a treatment for patients with COVID19 lung disease is based on two key facets of the disease: i) all patients presenting with symptoms have been infected with SARS-CoV-2 and the virus has gained entry into the airway cells, therefore, targeting the viral receptors to prevent cell entry may not a viable strategy to treat patients with this infection; and ii) Viruses are dependent on cellular proteins for each step of their life cycle and they hijack many of the host cell proteins for their function. Host kinase inhibitors represent one category of such proteins that can be targeted and have great potential to be repurposed as antivirals. Viruses hijack a large number of host kinases at distinct steps of their life cycle. Some of these host kinases are broadly required and thus represent attractive targets for broad-spectrum therapy. These findings, combined with the development and approval of a large number of kinase inhibitors for the treatment of cancer and inflammatory conditions have sparked efforts aimed to determine the therapeutic potential of such drugs to combat viral infections.

Disclosed herein are the results of a small molecule screen against SARS-CoV-2 live virus (strain Isolate USA-WA1/2020) using a library of small molecules to identify compounds which inhibited the infection, replication, or entry of the virus into the host cells. Out of a set of 430 hit compounds, a subset emerged that targets the m-tor, PIK3, ABL, and/or JAK pathways. Moreover, none of the cells treated with the hit compounds showed effects of toxicity.

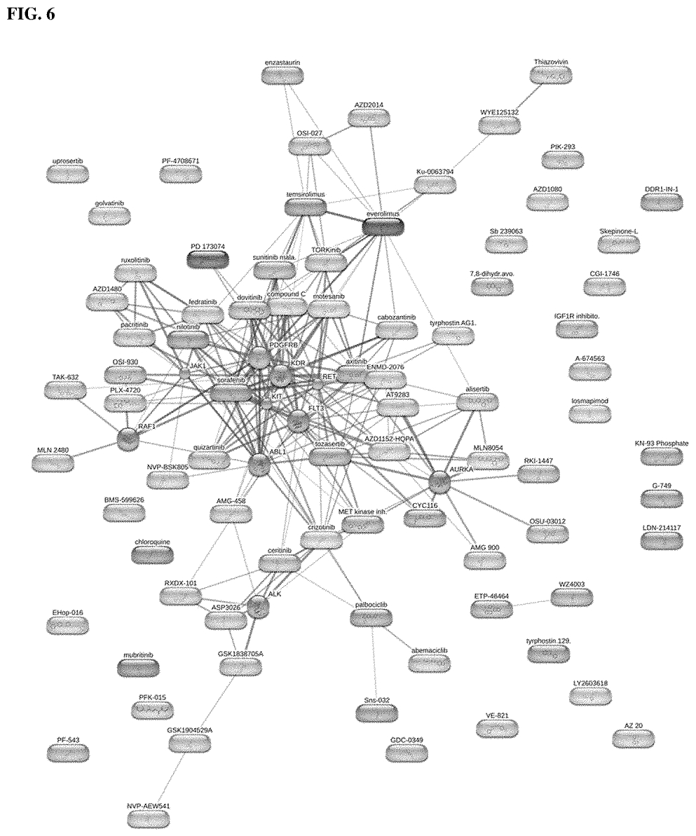

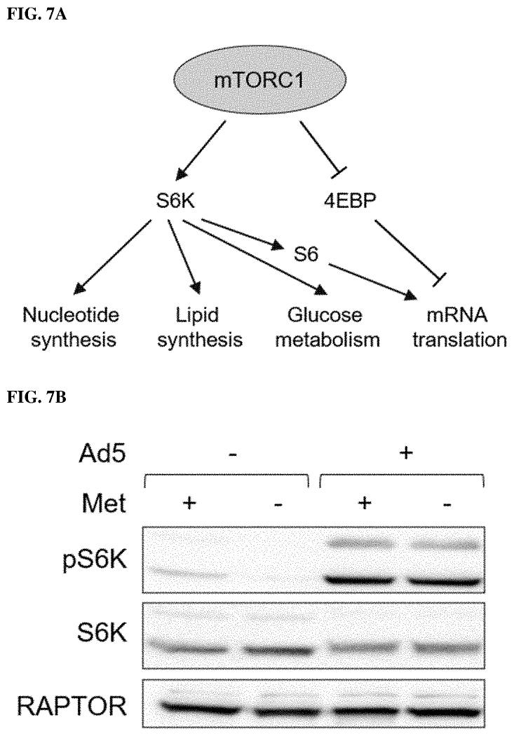

In addition, viruses increase anabolism in cells by activating mTORC1, a master regulator of anabolic metabolism (FIG. 7). mTORC1-driven anabolism metabolism aids viral replication by promoting nucleotide synthesis for RNA replication, protein synthesis for viral protein production, and lipid synthesis for viral envelop production. Indeed, SARS-CoV-2 activates mTORC1 in stem cell-derived human lung air liquid interface cultures from two different donors (FIG. 8). In view of the foregoing, it was hypothesized that SARS-CoV-2 could be susceptible to mTORC1 inhibition by drugs such as rapamycin, temsirolimus, and everolimus.

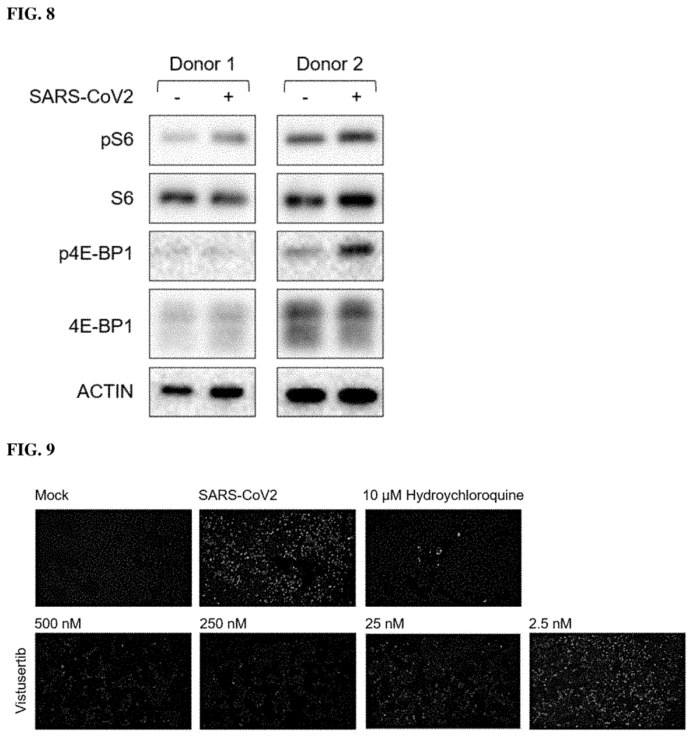

As a proof-of-concept experiment to show that mTORC1 inhibitors can reduce SARS-CoV-2 replication, Vero cells were treated with a range of doses of the mTORC1 inhibitor vistusertib (FIG. 9). Vehicle-treated cells showed high levels of SARS-CoV-2 dsRNA, whereas treatment with a low dose of vistusertib (25 nM) almost completely abolished the presence of viral dsRNA. Accordingly, disclosed herein are methods of treating viral infections (e.g., COVID-19).

In certain aspects, the present disclosure provides methods of treating a viral infection in a subject in need thereof, comprising administering a compound selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, an mTORC inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

In certain aspects, the present disclosure provides methods of treating a viral infection in a subject in need thereof, comprising administering a compound selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

In certain embodiments, the compound is PFK15, Uprosertib, LY3023414, A-674563, LDK378, ASP3026, dorsomorphin, WZ4003, AZ20, VE-821, Chloroquine, Berzosertib, Alisertib, Barasertib, VX-680, MLN8054, AMG-900, CYC116, Nilotinib, AT9283, CGI1746, KN-93, SNS-032, LY2835219, Palbociclib, LY2603618, Dovitinib, OSI-930, AMG-458, SU11274, Crizotinib, Golvatinib, DDR1-IN-1, AG-1478, FIIN-2, PD173074, Quizartinib, ENMD-2076, G-749, AZD1080, AC480, Mubritinib, GSK1838705A, NVP-AEW541, GSK1904529A, PQ 401, AZD1480, TG101348, NVP-BSK805, S-Ruxolitinib, Pacritinib, Everolimus, Temsirolimus, KU-0063794, AZD2014, WYE-125132, ETP-46464, OSI-027, GDC-0349, PP242, Losmapimod, Skepinone-L, SB239063, Tyrphostin AG 1296, OSU-03012, PIK-293, Enzastaurin, EHop-016, Sorafenib, MLN2480, PLX-4720, TAK-632, Thiazovivin, RKI-1447, PF-4708671, PF-543, LDN-214117, Entrectinib, 7,8-Dihydroxyflavone, Cabozantinib, Axitinib, Motesanib, or Sunitinib. In certain embodiments, the compound is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464. Ion certain embodiments, the compound is URMC-099, Torin2, SB525334, Salmeterol, Salbutamol, Rottlerin, Repsox, Rapamycin, Nilotinib, LY2109761, Galunisertib, Formoterol, Berzosertib, AZS8055, or Curculigoside. In the certain preferred embodiments, the compound is Berzosertib.

In certain embodiments, the methods disclosed herein further comprises administering an additional compound, wherein the additional compound inhibitor is selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3 CB inhibitor, a PIK3CD inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor. In certain embodiments, the additional compound is PFK15, Uprosertib, LY3023414, A-674563, LDK378, ASP3026, dorsomorphin, WZ4003, AZ20, VE-821, Chloroquine, Berzosertib, Alisertib, Barasertib, VX-680, MLN8054, AMG-900, CYC116, Nilotinib, AT9283, CGI1746, KN-93, SNS-032, LY2835219, Palbociclib, LY2603618, Dovitinib, OSI-930, AMG-458, SU11274, Crizotinib, Golvatinib, DDR1-IN-1, AG-1478, FIIN-2, PD173074, Quizartinib, ENMD-2076, G-749, AZD1080, AC480, Mubritinib, GSK1838705A, NVP-AEW541, GSK1904529A, PQ 401, AZD1480, TG101348, NVP-BSK805, S-Ruxolitinib, Pacritinib, Everolimus, Temsirolimus, KU-0063794, AZD2014, WYE-125132, ETP-46464, OSI-027, GDC-0349, PP242, Losmapimod, Skepinone-L, SB239063, Tyrphostin AG 1296, OSU-03012, PIK-293, Enzastaurin, EHop-016, Sorafenib, MLN2480, PLX-4720, TAK-632, Thiazovivin, RKI-1447, PF-4708671, PF-543, LDN-214117, Entrectinib, 7,8-Dihydroxyflavone, Cabozantinib, Axitinib, Motesanib, or Sunitinib. In certain embodiments, the additional compound is URMC-099, Torin2, SB525334, Salmeterol, Salbutamol, Rottlerin, Repsox, Rapamycin, Nilotinib, LY2109761, Galunisertib, Formoterol, Berzosertib, AZS8055, or Curculigoside. In certain embodiments, the additional compound, wherein the additional compound inhibitor is selected from MTOR, AKT1, PIK3, PIK3CB, PIK3CD, MAPK14, SRC, JAK2, ATR, MLK, BRAF(V600E/WT), Vps34, ABL1, or ABL2 inhibitor. In certain embodiments, the additional compound is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464.

In certain aspects, the present disclosure provides methods of treating a viral infection in a subject in need thereof, comprising administering a kinase inhibitor selected from an MTOR, mTORC, AKT1, PIK3, PIK3CB, PIK3CD, MAPK14, SRC, JAK2, ATR, MLK, BRAF(V600E/WT), Vps34, ABL1, or ABL2 inhibitor.

In certain aspects, the present disclosure provides methods of treating a viral infection in a subject in need thereof, comprising administering a kinase inhibitor selected from an MTOR, AKT1, PIK3, PIK3CB, PIK3CD, MAPK14, SRC, JAK2, ATR, MLK, BRAF(V600E/WT), Vps34, ABL1, or ABL2 inhibitor.

In certain embodiments, the kinase inhibitor is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464.

In certain embodiments, the methods disclosed herein further comprise administering an additional kinase inhibitor, wherein the additional kinase inhibitor is selected from MTOR, AKT1, PIK3, PIK3CB, PIK3CD, MAPK14, SRC, JAK2, ATR, MLK, BRAF(V600E/WT), Vps34, ABL1, or ABL2 inhibitor. In certain embodiments, the additional kinase inhibitor is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464.

In certain embodiments, the methods disclosed herein further comprise administering an additional kinase inhibitor, wherein the additional kinase inhibitor is selected from MTOR, AKT1, PIK3, PIK3CB, PIK3CD, MAPK14, SRC, JAK2, ATR, MLK, BRAF(V600E/WT), Vps34, ABL1, or ABL2 inhibitor.

In certain embodiments, the additional kinase inhibitor is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464.

In another aspect, the present disclosure provides methods of treating a viral infection in a subject in need thereof, comprising administering two or more compounds selected from URMC-099, Torin2, SB525334, Salmeterol, Salbutamol, Rottlerin, Repsox, Rapamycin, Nilotinib, LY2109761, Galunisertib, Formoterol, Berzosertib, AZS8055, or Curculigoside. In certain embodiments, the two compounds are LY2109761 and Nilotinib, Rottlerin and Nilotinib, URMC-099 and Nilotinib, Nilotinib and Rottlerin, AZS8055 and Rottlerin, Rapamycin and Nilotinib, Rottlerin and Nilotinib, Repsox and LY2109761, Berzosertib and Salmeterol, Repsox and Formoterol, Nilotinib and Salmeterol, Rottlerin and SB525334, URMC-099 and Repsox, URMC-099 and Nilotinib, Salbutamol and Salmeterol, Repsox and Salmeterol, Curculigoside and Salmeterol, Curculigoside and Galunisertib, LY2109761 and Rottlerin, Salmeterol and Rottlerin, Salbutamol and Rottlerin, Repsox and Rottlerin, or LY2109761 and Nilotinib.

In certain aspects, the present disclose provides methods of treating or preventing a viral infection in a subject in need thereof, comprising administering an mTORC inhibitor to the subject. In certain embodiments, the mTORC inhibitor is an mTORC1 inhibitor. In certain preferred embodiments, the mTORC inhibitor is vistusertib, rapamycin, temsirolimus, or everolimus.

In certain embodiments of the methods disclosed herein, the viral infection is selected from hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, and lower or upper respiratory tract infection (e.g., respiratory syncytial virus). In certain preferred embodiments, the viral infection is an influenza virus (e.g., the flu). In certain preferred embodiments, the viral infection is an influenza virus (e.g., the flu). In certain preferred embodiments, the viral infection is a common cold. In certain preferred embodiments, the coronavirus is SARS-CoV-2. In certain embodiments, the viral infection is COVID-19. In other embodiments, the viral infection is human cytomegalovirus, dengue virus, chikungunya virus, or nipah virus. In yet other embodiments, the viral infection is human cytomegalovirus, dengue virus, chikungunya virus, lassa virus, ebolavirus or nipah virus.

In certain embodiments of the methods disclosed herein, the compound or kinase inhibitor is administered via inhalation (e.g., via nasal or oral inhalation). In certain embodiments, the compound or kinase inhibitor is administered via oral inhalation. In other embodiments, the compound or kinase inhibitor is administered via nasal inhalation. In certain embodiments of the methods disclosed herein, the compound or kinase inhibitor is administered prophylactically.

In certain embodiments of the methods disclosed herein, the method prevents the viral infection.

In certain embodiments of the methods disclosed herein, the method further comprises testing the subject for the presence of the virus and administering the compound or kinase inhibitor if the subject tests positive for the presence of the virus. In certain embodiments, the compound or kinase inhibitor is administered within 1, 2, 3, 4, 5, 6, 7, 8, 9, or 10 days of the subject testing positive for the virus. In certain embodiments, the compound or kinase inhibitor is administered within 1 or 2 days of the subject testing positive for the virus. In certain embodiments, the compound or kinase inhibitor is administered within 1 day of the subject testing positive for the virus. In certain embodiments, the compound or kinase inhibitor is administered within 1, 2, 3, 4, 5, 6, 7, 8, 9, or 10 days of the subject's exposure to the virus. In certain embodiments, the compound or kinase inhibitor is administered within 1 or 2 days of the subject's exposure to the virus. In certain embodiments, the compound or kinase inhibitor is administered within 1 day of the subject's exposure to the virus. In certain embodiments, the kinase inhibitor is administered via inhalation (e.g., via nasal or oral inhalation). In certain embodiments, the inhibitor is administered via oral inhalation. In certain embodiments, the inhibitor is administered via nasal inhalation. In certain embodiments, the kinase inhibitor is administered prophylactically. In certain embodiments, the method further comprises testing the subject for the presence of the virus and administering the mTORC inhibitor if the subject tests positive for the presence of the virus. In certain embodiments, the mTORC inhibitor is administered within 1, 2, 3, 4, 5, 6, 7, 8, 9, or 10 days of the subject testing positive for the virus. In certain embodiments, the mTORC inhibitor is administered within 1 or 2 days of the subject testing positive for the virus. In certain embodiments, the mTORC inhibitor is administered within 1 day of the subject testing positive for the virus. In certain embodiments, the mTORC inhibitor is administered within 1, 2, 3, 4, 5, 6, 7, 8, 9, or 10 days of the subject's exposure to the virus. In certain embodiments, the mTORC inhibitor is administered within 1 or 2 days of the subject's exposure to the virus. In certain embodiments, the mTORC inhibitor is administered within 1 day of the subject's exposure to the virus.

In certain embodiments of the methods disclosed here, the method further comprises administering an additional anti-viral agent. In certain embodiments, the additional antiviral agent is selected from remdesivir, MitoQ, a cell-derived therapeutic exosome, and berzosertib. In certain embodiments, the additional antiviral agent is selected from remdesivir, MitoQ, and berzosertib. In certain preferred embodiments, the additional antiviral agent is berzosertib. In certain embodiments, the additional antiviral agent is selected from remdesivir, PF-07321332, or molnupiravir.

In another aspect, the present disclosure provides compositions comprising a compound and a pharmaceutically acceptable excipient, wherein the composition is formulated for inhalation; and the compound is selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, an mTORC inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

In another aspect, the present disclosure provides compositions comprising a compound and a pharmaceutically acceptable excipient, wherein the composition is formulated for inhalation; and the compound is selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

In certain embodiments, the compound is FK15, Uprosertib, LY3023414, A-674563, LDK378, ASP3026, dorsomorphin, WZ4003, AZ20, VE-821, Chloroquine, Berzosertib, Alisertib, Barasertib, VX-680, MLN8054, AMG-900, CYC116, Nilotinib, AT9283, CGI1746, KN-93, SNS-032, LY2835219, Palbociclib, LY2603618, Dovitinib, OSI-930, AMG-458, SU11274, Crizotinib, Golvatinib, DDR1-IN-1, AG-1478, FIIN-2, PD173074, Quizartinib, ENMD-2076, G-749, AZD1080, AC480, Mubritinib, GSK1838705A, NVP-AEW541, GSK1904529A, PQ 401, AZD1480, TG101348, NVP-BSK805, S-Ruxolitinib, Pacritinib, Everolimus, Temsirolimus, KU-0063794, AZD2014, WYE-125132, ETP-46464, OSI-027, GDC-0349, PP242, Losmapimod, Skepinone-L, SB239063, Tyrphostin AG 1296, OSU-03012, PIK-293, Enzastaurin, EHop-016, Sorafenib, MLN2480, PLX-4720, TAK-632, Thiazovivin, RKI-1447, PF-4708671, PF-543, LDN-214117, Entrectinib, 7,8-Dihydroxyflavone, Cabozantinib, Axitinib, Motesanib, or Sunitinib.

In another aspect, the present disclosure provides compositions comprising a kinase inhibitor and a pharmaceutically acceptable excipient, wherein the composition is formulated for inhalation; and the kinase inhibitor is selected from an MTOR, mTORC AKT1, PIK3, PIK3CB, PIK3CD, MAPK14, SRC, JAK2, ATR, MLK, BRAF(V600E/WT), Vps34, ABL1, or ABL2 inhibitor.

In another aspect, the present disclosure provides compositions comprising a kinase inhibitor and a pharmaceutically acceptable excipient, wherein the composition is formulated for inhalation; and the kinase inhibitor is selected from an MTOR, AKT1, PIK3, PIK3CB, PIK3CD, MAPK14, SRC, JAK2, ATR, MLK, BRAF(V600E/WT), Vps34, ABL1, or ABL2 inhibitor. In certain embodiments, the kinase inhibitor is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464.

In another aspect, the present disclosure provides compositions comprising two or more compounds selected from URMC-099, Torin2, SB525334, Salmeterol, Salbutamol, Rottlerin, Repsox, Rapamycin, Nilotinib, LY2109761, Galunisertib, Formoterol, Berzosertib, AZS8055, or Curculigoside, and a pharmaceutically acceptable excipient. In certain embodiments, the two compounds are LY2109761 and Nilotinib, Rottlerin and Nilotinib, URMC-099 and Nilotinib, Nilotinib and Rottlerin, AZS8055 and Rottlerin, Rapamycin and Nilotinib, Rottlerin and Nilotinib, Repsox and LY2109761, Berzosertib and Salmeterol, Repsox and Formoterol, Nilotinib and Salmeterol, Rottlerin and SB525334, URMC-099 and Repsox, URMC-099 and Nilotinib, Salbutamol and Salmeterol, Repsox and Salmeterol, Curculigoside and Salmeterol, Curculigoside and Galunisertib, LY2109761 and Rottlerin, Salmeterol and Rottlerin, Salbutamol and Rottlerin, Repsox and Rottlerin, or LY2109761 and Nilotinib.

In another aspect, the present disclosure provides compositions comprising an mTORC inhibitor and a pharmaceutically acceptable excipient, wherein the composition is formulated for inhalation. In certain embodiments, the mTORC inhibitor is an mTORC1 inhibitor. In certain preferred embodiments, the mTORC inhibitor is vistusertib, rapamycin, temsirolimus, or everolimus.

In certain embodiments of the compositions disclosed herein, the composition is formulated for nasal inhalation. In other embodiments, the composition is formulated for oral inhalation.

In another aspect, the present disclosure provides an inhaler comprising a composition of the disclosure. In certain embodiments, the inhaler is a nasal inhaler. In other embodiments, the inhaler is an oral inhaler.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, Calif. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as an infection, a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer or an infection includes, for example, reducing the number of detectable cancerous growths or infections in a population of patients receiving a prophylactic treatment relative to an untreated control population, and/or delaying the appearance of detectable cancerous growths or infections in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds of the disclosure. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Screening of Exemplary Kinase Inhibitors

### Materials And Methods

### Virus and Cells

SARS-Related Coronavirus 2 (SARS-CoV-2), Isolate USA-WA1/2020, was obtained from BEI Resources of National Institute of Allergy and Infectious Diseases (NIAID). All the studies involving live virus were conducted in the UCLA BSL3 high-containment facility. SARS-CoV-2 was passaged once in Vero-E6 cells and viral stocks were aliquoted and stored at −80° C. Virus titer was measured in Vero-E6 cells by TCID₅₀ assay. Cells were cultured in DMEM growth media containing 10% fetal bovine serum (FBS), 2 mM L-glutamine, penicillin (100 units/ml), streptomycin (100 units/ml), and 10 mM HEPES. Cells were incubated at 37° C. with 5% CO₂.

### SARS-CoV2 Infection (General Methodology)

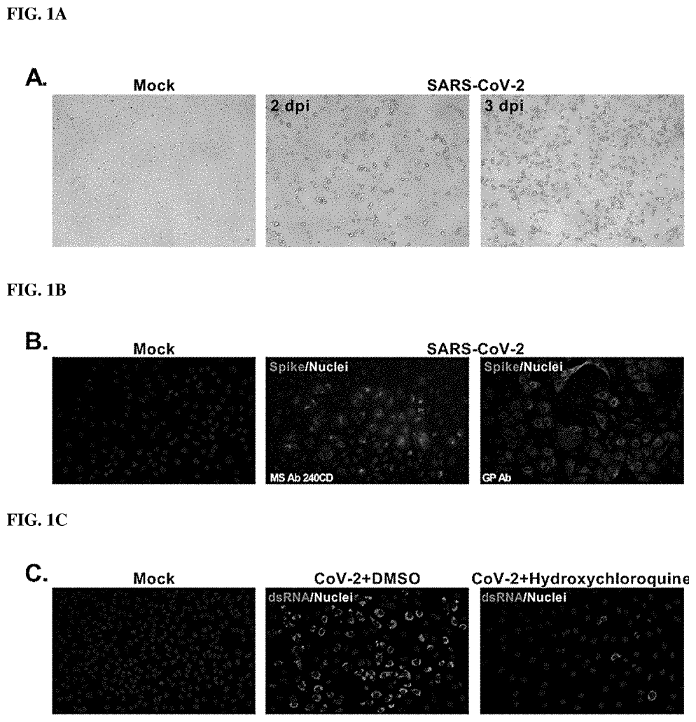

Vero cells were seeded at 1×10⁵ cells per well in 2 ml volumes using a 12-well plate. The following day, viral inoculum (MOI of 0.01 and 0.1; 400 μl/well) was prepared using serum free media. The spent media from each well was removed and 400 μl of prepared inoculum was added onto Vero cells. For mock infection, serum free media (400 μl/well) alone was added. The inoculated plates were incubated for 1 hr at 37° C. with 5% CO₂. The inoculum was spread by gently tilting the plate sideways at every 15 minutes. At the end of incubation, the inoculum was replaced with serum supplemented media (2 ml per well). At selected timepoints live cell images were obtained by bright field microscope. Striking cytopathic effect (CPE) was observed in SARS-CoV-2 infected cells (FIG. 1A), indicating viral replication and associated cell injury. At 48 hours post infection (hpi), viral infection was examined by immunocytochemistry (ICC) analysis using SARS-CoV Spike (S) antibodies [BEI Resources: NR-10361 Polyclonal Anti-SARS Coronavirus (antiserum, Guinea Pig), and NR-616 Monoclonal Anti-SARS-CoV S Protein (Similar to 240C) SARS coronavirus]. SARS-CoV spike antigen was detected in the cytoplasm of the infected cells, revealing active viral infection (FIG. 1B).

### Antiviral Drug Study

Once the SARS-CoV-2 infection system was established, the effect of the drug hydroxychloroquine (HQ; 10 um concentration) was evaluated. The cells seeded in chamber slides were pretreated with HQ for 1 hour, then SARS-CoV-2 inoculum (MOI 1) was added. DMSO vehicle treated cells, with or without viral infections, were included as controls. 48 hpi, the cells were fixed and immunostained with anti-dsRNA antibody (J2 clone; Absolute Antibody Inc, USA) to assess viral genome replication (FIG. 1C). HQ is a known endosome acidification inhibitor, which has effectively blocked SARS-CoV-2 infection. Taken together, these results establish various virological assays for investigating SARS-CoV-2 replicatory mechanisms, cell injury processes as well as evaluating antiviral agents.

### Protocol

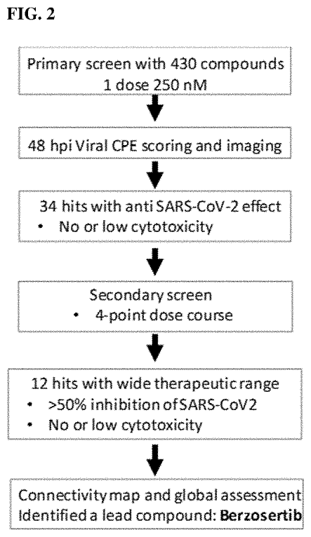

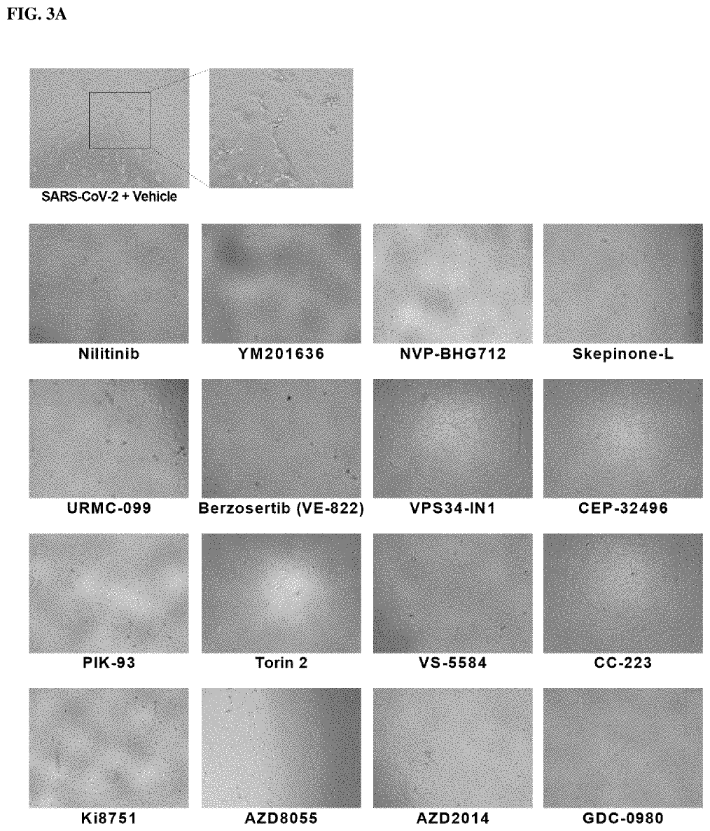

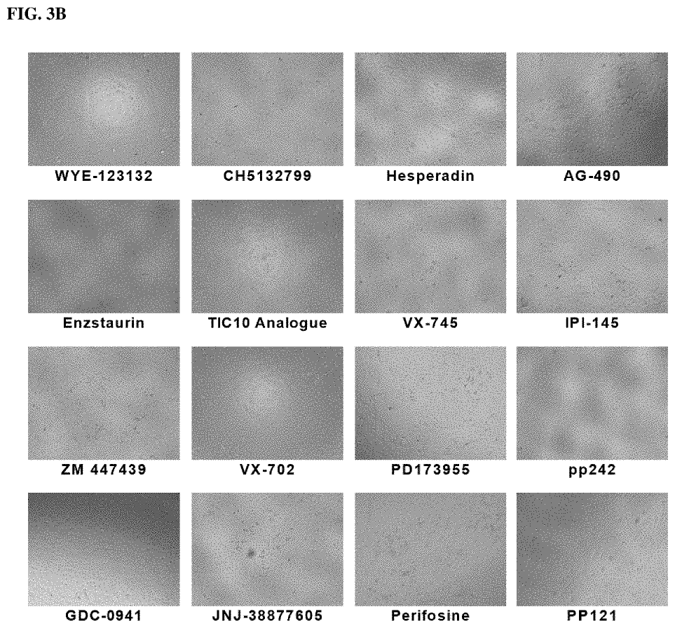

A step by step drug screen work flow is provided in FIG. 2. The drug compound library was selected based on the screening concentration that would have optimal activity and minimal toxicity. A set of 430 kinase inhibitors that are normally used for oncology and which also have Phase I/II/III data attached was tested. Compounds were formulated into DMSO and pre-plated into media at a 2× concentration (final drug concentration 250 nM). Compounds were added to the Vero cells in the BSL-3 lab and then virus (Multiplicity of Infection 0.1). After the 48 hour incubation at 37° C., 5% CO2, viral cytopathic effect was scored and imaged (FIGS. 3A & B). The compounds prevented the viral CPE were identified (FIGS. 3A & B, and Table 1) and subjected to pathway analysis. The compounds hit few selected kinases and a very limited set of pathways (FIG. 4), suggesting the specific nature of the antiviral agents.

TABLE 1
Exemplary Kinase Inhibitors

| | |
|---|---|
| 1 | Nilotinib (AMN-107) |
| 2 | ZM 447439 |
| 3 | JNJ-38877605 |
| 4 | AG-490 (Tyrphostin B42) |
| 5 | ZSTK474 |
| 6 | Enzastaurin (LY317615) |
| 7 | YM201636 |
| 8 | GDC-0941 |
| 9 | Hesperadin |
| 10 | PP242 |
| 11 | VX-745 |
| 12 | PIK-93 |
| 13 | NVP-BHG712 |
| 14 | Ki8751 |
| 15 | AZD8055 |
| 16 | AZD2014 |
| 17 | GDC-0980 (RG7422) |
| 18 | AS-604850 |
| 19 | WYE-125132 (WYE-132) |
| 20 | CH5132799 |
| 21 | Torin 2 |
| 22 | Skepinone-L |
| 23 | URMC-099 |
| 24 | TIC10 Analogue |
| 25 | VS-5584 (SB2343) |
| 26 | IPI-145 (INK1197) |
| 27 | VE-822 |
| 28 | VX-702 |
| 29 | PD173955 |
| 30 | CC-223 |
| 31 | VPS34-IN1 |
| 32 | CEP-32496 |
| 33 | Perifosine (KRX-0401) |
| 34 | PP121 |
| 35 | ETP-46464 |
| — | — |

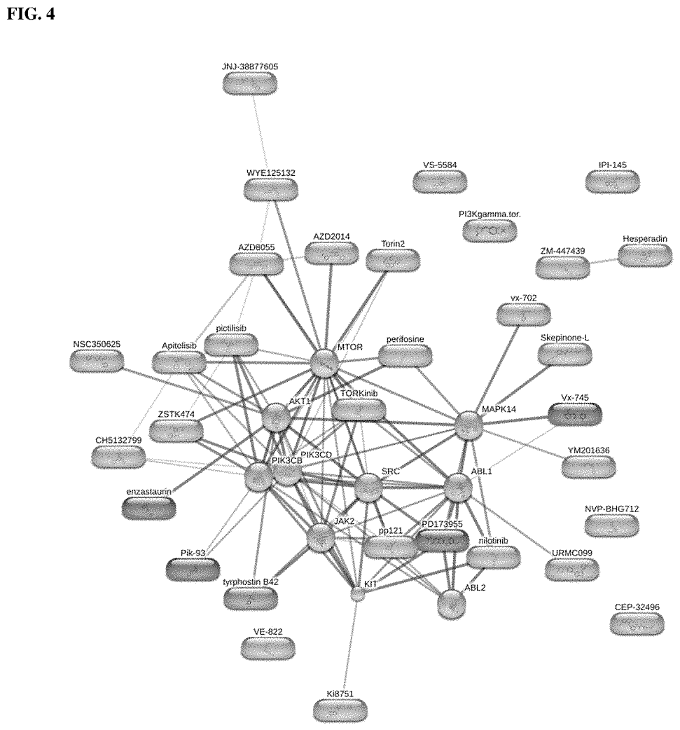

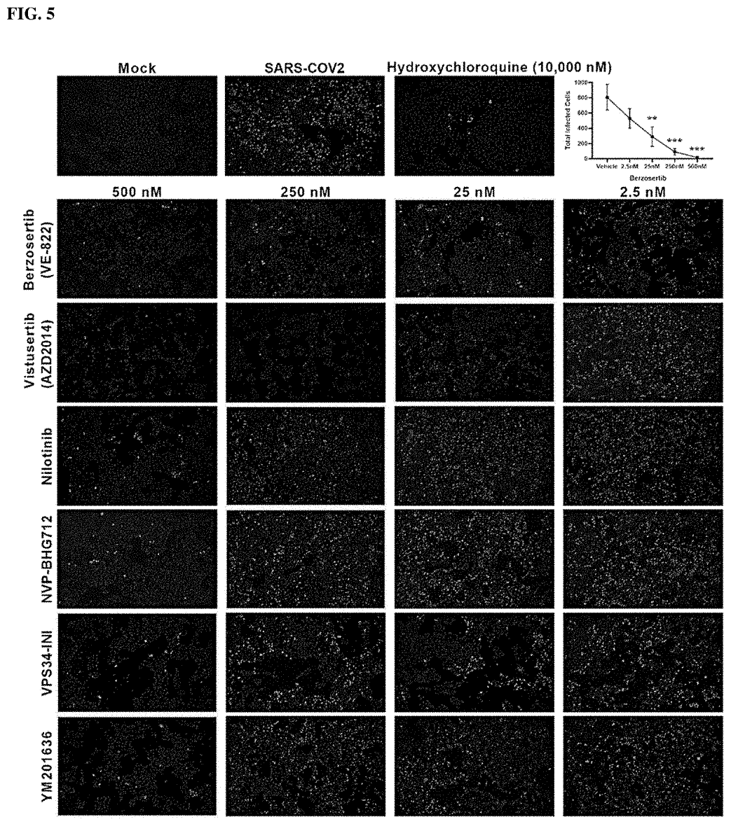

From the primary screen, 34 compounds were selected for secondary screen with multiple drug doses (2.5, 25, 250 and 500 nM). With sensitive immunofluorescent assay and quantification of viral infected cells verified many hits from primary screen (FIG. 5). Berzosertib (VE-822) and Vistusertib (AZD2014) antiviral activities are at IC₅₀ of 25 nM. Nilotinib, NVP-BHG712, VPS34-INI and YM201636 have IC₅₀ ranges between 250 nM-500 nM. These compounds exhibit as an antiviral agent against SARS-CoV-2 by limiting viral replication and cell death by directly acting or indirectly acting to inhibit critical cellular enzymes needed for viral replication. These kinase inhibitors are nucleoside analogues.

### Example 2: Screening of Exemplary Compounds

Using the same protocol as Example 1, URMC-099, Torin2, SB525334, Salmeterol, Salbutamol, Rottlerin, Repsox, Rapamycin, Nilotinib, LY2109761, Galunisertib, Formoterol, Berzosertib, AZS8055, and Curculigoside were tested combinatorially with doses ranging from 5000, 500, 50 and 5 nM of drug/component 1 vs 5000, 500 and 50 nM of drug/component 2. Below is a list of the most potent combinations as scored by saving cells from infections as indicated by surviving cell number (higher is better).

| | Compound A-Compound B | | |
|---|---|---|---|
| | | | Cell Number: |
| | Compound | Compound | Cell Nuclei |
| | A: (nM) | B: (nM) | Counts |
| LY2109761-Nilotinib | 50 | 50 | 11897 |
| Rottlerin-Nilotinib | 5 | 50 | 11139 |
| URMC-099-Nilotinib | 50 | 50 | 11058 |
| Nilotinib-Rottlerin | 50 | 50 | 10480 |
| Nilotinib-Rottlerin | 5 | 50 | 9690 |
| AZS8055-Rottleri | 50 | 50 | 9532 |
| Rapamycin-Nilotinib | 50 | 50 | 9138 |
| Rottlerin-Nilotinib | 50 | 50 | 9122 |
| Repsox-LY2109761 | 5 | 50 | 8936 |
| Berzosertib-Salmeterol | 50 | 50 | 8833 |
| Repsox-Formoterol | 5 | 50 | 8795 |
| Nilotinib-Salmeterol | 5 | 50 | 8638 |
| Rottlerin-SB525334 | 5 | 50 | 8601 |
| URMC-099-Repsox | 5 | 50 | 8553 |
| URMC-099-Nilotinib | 5 | 50 | 8518 |
| Empty-Formoterol | 50 | 50 | 8390 |
| Salbutamol-Salmeterol | 5 | 50 | 8383 |
| Repsox-Salmeterol | 5 | 50 | 8287 |
| Curculigoside-Salmeterol | 50 | 50 | 8281 |
| Curculigoside-Galunisertib | 5 | 50 | 8263 |
| LY2109761-Rottlerin | 5 | 50 | 8196 |
| Empty-Salmeterol | 50 | 50 | 8193 |
| Salmeterol-Rottlerin | 50 | 50 | 8148 |
| Salbutamol-Rottlerin | 5 | 50 | 8146 |
| Repsox-Rottlerin | 50 | 50 | 8017 |
| LY2109761-Nilotinib | 5 | 50 | 8014 |

### Example 3: Testing of an Exemplary Kinase Inhibitor in Air-Liquid Interface (Ail) Cells

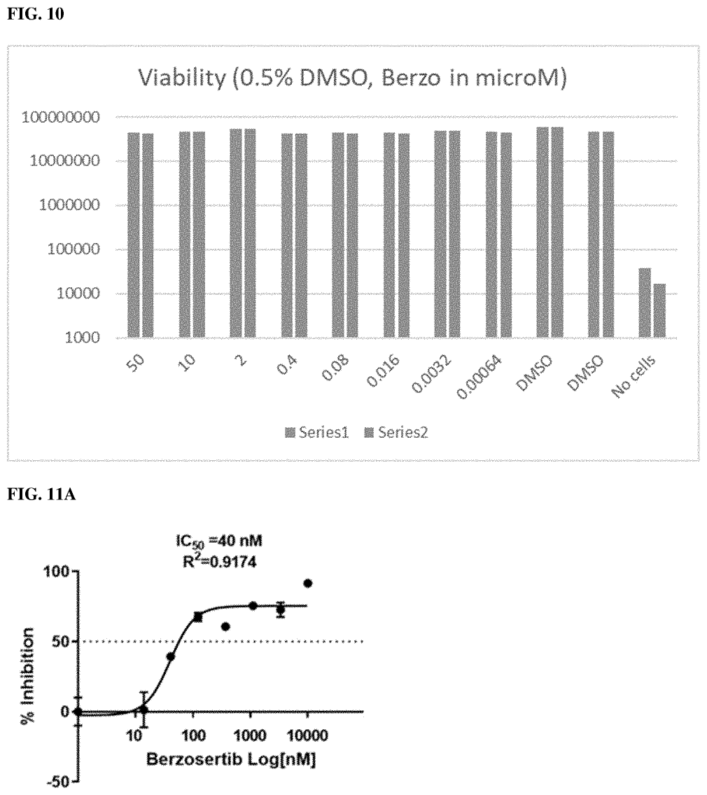

Berzosertib is non-toxic to human lung stem cell derived Air-liquid interface (ALI) cells. Berzosertib was tested in a primary human lung tissue culture system consisting of mucociliary air-liquid interface (ALI) cultures derived from primary human proximal airway basal stem cells (ABSCs). 24-well 6.5 mm trans-wells with 0.4 mm pore polyester membrane inserts were used for culturing ALI cells. 500 μl ALI media (PneumaCult-ALI Medium, STEMCELL Technologies) was used in the basal chamber for ALI cultures and cells were cultured at 37° C. with 5% CO₂. The indicated concentrations (50, 10, 2, 0.4, 0.08, 0.0016, 0.0032, 0.00064 μM) of berzosertib was added in the basal chamber. Control cells were treated with the solvent DMSO. 48 hours post drug treatment, the cells were subjected to viability assay using Cell Titer glo kit. Berzosertib treatment did not have any deleterious effect on cell viability that is similar to control DMSO treated cells (FIG. 10). In conclusion, in human ALI lung cells, berzosertib is safe even at the highest tested concentration of 50 μM.

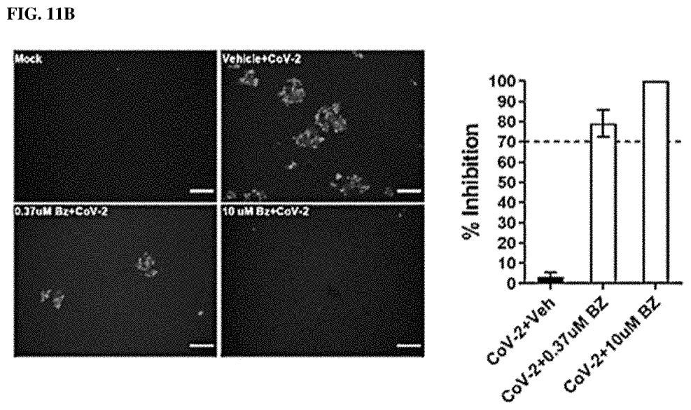

The experiment was repeated with various concentrations of berzosertib treatment in ALI cells. At 48 hours post infection (hpi) the cells were collected for RNA and also fixed with 4% PFA. Viral infection was examined by RTQPCR and immunocytochemistry (ICC) analysis using SARS-CoV Spike (S) antibody [BEI Resources: NR-616 monoclonal anti-SARS-CoV S protein (Similar to 240C) SARS coronavirus]. Also in this ALI experiment, berzosertib was effective in inhibiting SARS-CoV-2 at an IC₅₀ of 40 nM concentration determined by RTQPCR and over 70% inhibition of virus positive cells (FIGS. 11A and 11B).

These results demonstrate that berzosertib is non-toxic in ALI cells even at high 50 μM concentration with an IC50 at nanomolar concentration (FIGS. 10 and 11), which gives over 200 fold therapeutic window.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating a viral infection in a subject in need thereof, comprising administering a compound selected from a PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, an mTORC inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

2. (canceled)

3. The method of claim 1, wherein the compound is PFK15, Uprosertib, LY3023414, A-674563, LDK378, ASP3026, dorsomorphin, WZ4003, AZ20, VE-821, Chloroquine, Berzosertib, Alisertib, Barasertib, VX-680, MLN8054, AMG-900, CYC116, Nilotinib, AT9283, CGI1746, KN-93, SNS-032, LY2835219, Palbociclib, LY2603618, Dovitinib, OSI-930, AMG-458, SU11274, Crizotinib, Golvatinib, DDR1-IN-1, AG-1478, FIIN-2, PD173074, Quizartinib, ENMD-2076, G-749, AZD1080, AC480, Mubritinib, GSK1838705A, NVP-AEW541, GSK1904529A, PQ 401, AZD1480, TG101348, NVP-BSK805, S-Ruxolitinib, Pacritinib, Everolimus, Temsirolimus, KU-0063794, AZD2014, WYE-125132, ETP-46464, OSI-027, GDC-0349, PP242, Losmapimod, Skepinone-L, SB239063, Tyrphostin AG 1296, OSU-03012, PIK-293, Enzastaurin, EHop-016, Sorafenib, MLN2480, PLX-4720, TAK-632, Thiazovivin, RKI-1447, PF-4708671, PF-543, LDN-214117, Entrectinib, 7,8-Dihydroxyflavone, Cabozantinib, Axitinib, Motesanib, or Sunitinib.

4. The method of claim 1, wherein the compound is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464.

5. The method of claim 1, wherein the compound is URMC-099, Torin2, SB525334, Salmeterol, Salbutamol, Rottlerin, Repsox, Rapamycin, Nilotinib, LY2109761, Galunisertib, Formoterol, Berzosertib, AZS8055, or Curculigoside.

6. The method of claim 1, wherein the method further comprises administering an additional compound, wherein the additional compound inhibitor is selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

7. The method of claim 1, wherein the additional compound is PFK15, Uprosertib, LY3023414, A-674563, LDK378, ASP3026, dorsomorphin, WZ4003, AZ20, VE-821, Chloroquine, Berzosertib, Alisertib, Barasertib, VX-680, MLN8054, AMG-900, CYC116, Nilotinib, AT9283, CGI1746, KN-93, SNS-032, LY2835219, Palbociclib, LY2603618, Dovitinib, OSI-930, AMG-458, SU11274, Crizotinib, Golvatinib, DDR1-IN-1, AG-1478, FIIN-2, PD173074, Quizartinib, ENMD-2076, G-749, AZD1080, AC480, Mubritinib, GSK1838705A, NVP-AEW541, GSK1904529A, PQ 401, AZD1480, TG101348, NVP-BSK805, S-Ruxolitinib, Pacritinib, Everolimus, Temsirolimus, KU-0063794, AZD2014, WYE-125132, ETP-46464, OSI-027, GDC-0349, PP242, Losmapimod, Skepinone-L, SB239063, Tyrphostin AG 1296, OSU-03012, PIK-293, Enzastaurin, EHop-016, Sorafenib, MLN2480, PLX-4720, TAK-632, Thiazovivin, RKI-1447, PF-4708671, PF-543, LDN-214117, Entrectinib, 7,8-Dihydroxyflavone, Cabozantinib, Axitinib, Motesanib, or Sunitinib.

8. The method of claim 1, wherein the method further comprises administering an additional compound, wherein the additional compound is URMC-099, Torin2, SB525334, Salmeterol, Salbutamol, Rottlerin, Repsox, Rapamycin, Nilotinib, LY2109761, Galunisertib, Formoterol, Berzosertib, AZS8055, or Curculigoside.

9. The method of claim 1, wherein the method further comprises administering an additional compound, wherein the additional compound inhibitor is selected from MTOR, AKT1, PIK3, PIK3CB, PIK3CD, MAPK14, SRC, JAK2, ATR, MLK, BRAF(V600E/WT), Vps34, ABL1, or ABL2 inhibitor.

10. The method of claim 1, wherein the additional compound is Nilotinib (AMN-107), ZM 447439, JNJ-38877605, AG-490 (Tyrphostin B42), ZSTK474, Enzastaurin (LY317615), YM201636, GDC-0941, Hesperadin, PP242, VX-745, PIK-93, NVP-BHG712, Ki8751, AZD8055, AZD2014, GDC-0980 (RG7422), AS-604850, WYE-125132 (WYE-132), CH5132799, Torin 2, Skepinone-L, URMC-099, TIC10 Analogue, VS-5584 (SB2343), IPI-145 (INK1197), VE-822, VX-702, PD173955, CC-223, VPS34-IN1, CEP-32496, Perifosine (KRX-0401), PP121, or ETP-46464.

11-18. (canceled)

19. The method of claim 1, wherein the mTORC inhibitor is an mTORC1 inhibitor.

20. The method of claim 1, wherein the mTORC inhibitor is vistusertib, rapamycin, temsirolimus, or everolimus.

21. The method of claim 1, wherein the viral infection is selected from hepatitis C, norovirus, junin, dengue virus, coronavirus, human immunodeficiency virus, herpes simplex, avian flu, chickenpox, cold sores, common cold, glandular fever, influenza, measles, mumps, pharyngitis, pneumonia, rubella, severe acute respiratory syndrome, respiratory syncytial virus, human cytomegalovirus, dengue virus, chikungunya virus, lassa virus, ebolavirus and nipah virus.

22-24. (canceled)

25. The method of claim 1, wherein the viral infection is COVID-19.

26. (canceled)

27. The method of claim 1, wherein the compound is administered via inhalation.

28. (canceled)

29. (canceled)

30. The method of claim 1, wherein the compound is administered prophylactically.

31. (canceled)

32. The method of claim 1, wherein the method further comprises testing the subject for the presence of the virus and administering the compound if the subject tests positive for the presence of the virus.

33. (canceled)

34. The method of claim 32, wherein the compound is administered within 1 or 2 days of the subject testing positive for the virus.

35. (canceled)

36. (canceled)

37. The method of claim 1, wherein the compound is administered within 1 or 2 days of the subject's exposure to the virus.

38-53. (canceled)

54. A composition comprising a compound and a pharmaceutically acceptable excipient, wherein the composition is formulated for inhalation; and the compound is selected from an PFKFB3 inhibitor, a CDK inhibitor, a PKA inhibitor, an ALK inhibitor, an AMPK inhibitor, an ATM inhibitor, an aurora kinase inhibitor, an VEGFR inhibitor, an Bcr-Abl inhibitor, a BTK inhibitor, a CaMK inhibitor, a Chk inhibitor, a c-Kit inhibitor, an FGFR inhibitor, an Flt inhibitor, an VEGFR inhibitor, an PDGFR inhibitor, a c-Met inhibitor, a DDR inhibitor, a FLT3 inhibitor, a GSK-3 inhibitor, a HER2 inhibitor, an IGF-1 inhibitor, an IGF-1R inhibitor, a mTOR inhibitor, an mTORC inhibitor, a p38 inhibitor, an MAPK inhibitor, a PDK-1 inhibitor, a PKC inhibitor, a Rac inhibitor, a Raf inhibitor, a ROCK inhibitor, a S6 kinase inhibitor, an SphK1 inhibitor, a TGF-beta inhibitor, a Smad inhibitor, a Trk inhibitor, a Tie-2 inhibitor, an MTOR inhibitor, an AKT inhibitor, an AKT1 inhibitor, a PIK3 inhibitor, a PIK3CB inhibitor, a PIK3CD inhibitor, an mTORC inhibitor, a MAPK14 inhibitor, an SRC inhibitor, a JAK inhibitor, a JAK2 inhibitor, an ATR inhibitor, a MLK inhibitor, a BRAF(V600E/WT) inhibitor, a Vps34 inhibitor, a ABL1 inhibitor, and an ABL2 inhibitor.

55-66. (canceled)

67. An inhaler comprising the composition of claim 54.

68. (canceled)

69. (canceled)
